(19) United States
(12) Patent Application Publication
(10) Pub. No.: US 2011/0104673 A1
(43) Pub. Date: May 5, 2011

(54) NUCLEIC ACID SEQUENCES AND METHODS FOR IDENTIFYING COMPOUNDS THAT AFFECT RNA/RNA BINDING PROTEIN INTERACTIONS AND mRNA FUNCTIONALITY

(76) Inventors: ANTHONY GIORDANO, PHOENIXVILLE, PA (US); ASISH XAVIER, WEST CHESTER, PA (US)

(21) Appl. No.: 12/755,236

(22) Filed: Apr. 6, 2010

Related U.S. Application Data
(63) Continuation of application No. 11/414,983, filed on May 1, 2006, now abandoned.
Application No. 11/414,983 is a continuation of application No. 10/963,062, filed on Oct. 12, 2004, now abandoned.
Application No. 10/963,062 is a continuation of application No. 09/437,458, filed on Nov. 10, 1999, now abandoned.

(51) Int. Cl. C12Q 1/68; C07H 21/04; C07H 21/02; G01N 33/53

(52) U.S. Cl. 435/6; 536/23.1; 436/94; 436/501

(57) ABSTRACT

Disclosed herein are nucleic acid sequences and their optimized subfragments which are located in the mRNA untranslated regions of therapeutically-relevant genes. These sequences specifically bind RNA binding proteins (RBPs) and/or regulate the mRNA functionality. Also disclosed are methods of optimizing a subfragment of a parent nucleic acid sequence such that the RBP binding activity or mRNA functionality of the parent nucleic acid sequence is preserved in the optimized subfragment.

### FIELD OF THE INVENTION

This invention relates to the field of nucleic acid regulatory elements to that affect post-transcriptional regulation of protein expression. More specifically, the invention features nucleic acid sequences, located in the mRNA untranslated regions of various therapeutically-relevant genes which specifically bind RNA binding proteins (RBPs) or have other roles in the regulation of protein expression from the linked RNA. These sequences are used in screening assays, for example, to identify compounds that affect the RNA/RBP binding pair interaction. Such compounds can be administered therapeutically to regulate protein expression.

### BACKGROUND OF THE INVENTION

The regulation of protein expression can occur at a number of levels: transcriptional, post-transcriptional, or post-translational. The modulation of protein expression can produce significant benefits in the treatment of disease. Accordingly, recent efforts to achieve such regulatory control have focused on the development of small molecules that regulate transcription factors as well as antisense molecules that inhibit protein expression. While the transcription factor approach holds promise, there are drawbacks. For example, a compound which modulates a transcription factor may lack specificity and require nuclear localization; both of these limitations are problematic for those in the field of drug development. For example, a drug that affects the binding of a targeted transcription factor and has beneficial results may also affect transcription of many other genes in a deleterious manner. In addition, there are difficulties in designing a drug that both effectively interacts with the target and is transported into the nucleus. Targeting RNA with antisense oligonucleotides to inhibit protein expression also has drawbacks; the approach is restricted to strategies for decreasing protein expression and may be limited by relatively poor intracellular transport of oligonucleotides.

Another possible approach to modifying post-transcriptional protein expression in eukaryotic cells involves targeting the specific interaction of proteins that bind RNA (RNA binding proteins or RBPs) with the RNA molecules. These RBPs appear to mediate the processing of pre-mRNAs, the transport of mRNA from the nucleus to the cytoplasm. mRNA stabilization, translational efficiency, and the sequestration of some mRNAs. The most common RBP motifs are the RNP motif, the Arg-rich motif, the RGG box, the KH motif, and the double-stranded RNA-binding motif (Burd and Dreyfuss, Science 265: 615-621, 1994). Some key factors in RNA/RBP interactions involve sequence and structure recognition, positional effects, and pre-binding to Z-DNA (Herbert et al., Proc. Natl. Acad. Sci. USA 92: 7550-7554, 1995; Malter 246: 664-666, 1989).

Nucleic acid sequences and screening methods for detecting compounds which alter the RNA/RBP binding pair interaction would greatly speed the discovery of therapeutic drugs which alter protein expression.

### SUMMARY OF THE INVENTION

In general, the invention features nucleic acid sequences and their optimized subfragments that are located in the mRNA untranslated regions of various, therapeutically-relevant genes. These sequences specifically bind RNA binding proteins (RBPs) and or are involved in mRNA functionality. Also featured are methods of truncating parent sequences to create optimized subfragments. These parent sequences and optimized sequences are used as RNA molecule targets to screen for compounds that affect the RNA/RBP binding pair interaction or mRNA functionality. Compounds identified by the screening process can be administered therapeutically to regulate protein expression.

In a first aspect, the invention features a nucleic acid sequence comprising one of the nucleic acid sequences of SEQ ID NOs 1-20, or a subfragment nucleic acid sequence of SEQ ID NOs 1-20, wherein an mRNA to molecule comprising the sequence has RNA binding protein (RBP) binding activity or regulates the functionality of the mRNA. These nucleic acid sequences are derived from the following gene sequences: the human interleukin-6 receptor, Accession #'s X12830, M20556 (SEQ ID NO 1); human cyclooxygenase-2, Accession #M90100 (SEQ ID NO 2 and SEQ ID NO 11); human ubiquitin, Accession #U49869, X04803 (SEQ ID NO 3); human uracil DNA glycosylase-1 (Ung-1), Accession #X89398 (SEQ ID NO 4); human excision repair controlling gene, Accession #U16815 (SEQ ID NO 5); human cytochrome C oxidase 6b, Accession #D28426 (SEQ ID NO 6); human cytochrome C oxidase 5b, Accession #U41284 (SEQ ID NO 7); human Beta-2 adrenergic receptor, Accession #M15169, J02728, M16106 (SEQ ID NO 8); human breast cancer susceptibility-2 (BRCA2), Accession #U43746 (SEQ ID NO 9); human interleukin-4, Accession #M23442 (SEQ ID NO 10); human vascular cell adhesion molecule-1, Accession #M73255 (SEQ ID NO 12 and SEQ ID NO 13); human interleukin-11, Accession #81890 (SEQ ID NO 14); human macrophage CSF receptor (c-fms proto-oncogene), Accession #X03663 (SEQ ID NO 15); human interleukin-9, Accession #M86593, M55519 (SEQ ID NO 16); human leptin (murine obesity homologue), Accession #NM_{—}000230 (SEQ ID NO 17); rat neuropeptide Y5 receptor, Accession #U66274 (SEQ ID NO 18); rat orexin receptor-1, Accession #AF0411244 (SEQ ID NO 19); and mouse mahogany protein, Accession #AF116897 (SEQ ID NO 20). Preferably, the subfragment nucleic acid sequence is optimized. Preferably, the mRNA functionality of the parent or subfragment sequence involves an alteration in pre-mRNA processing or in the stabilization, translational efficiency, localization, sequestration, editing, or splicing functions of the UTR-associated mRNA, more preferably, the alteration in pre-mRNA processing or in the stabilization, translational efficiency, localization, sequestration, editing, or splicing functions of the UTR-associated mRNA comprises a change of at least 20% above or below the corresponding value for a control mRNA that lacks the sequence.

In a second aspect, the invention provides a method of identifying an optimized subfragment of any one of the parent nucleic acid sequences of SEQ ID NOs 1-20, wherein the method involves isolating a subfragment nucleic acid sequence from the parent nucleic acid sequence, assaying RNA molecules comprising the subfragment for RBP binding activity or mRNA functionality, and identifying a subfragment nucleic sequence that maintains an RBP binding activity and/or mRNA functionality that is equivalent to the parent sequence. Preferably, the subfragment nucleic acid sequence is isolated by restriction enzyme digestion, and the subfragment is identified by deletion mapping. Preferably, the mRNA functionality of the sequences involves an alteration in pre-mRNA processing or in the stabilization, translational efficiency, localization, sequestration, editing, or splicing functions of the UTR-associated mRNA, more preferably, the alteration in pre-mRNA processing or in the stabilization, translational efficiency, localization, sequestration, editing, or splicing functions of the UTR-associated mRNA comprises a change of at least 20% above or below the corresponding value for a control mRNA that lacks the sequence.

A third and related aspect of the invention provides a nucleic acid sequence identified as an optimized subfragment of any one of SEQ ID NOs 1-20 by a method which involves isolating a subfragment nucleic acid sequence from the parent nucleic acid sequence, assaying RNA molecules comprising the subfragment for RBP binding activity or mRNA functionality, and identifying a subfragment nucleic sequence that maintains an RBP binding activity and/or mRNA functionality that is equivalent to the parent sequence. Preferably, the to mRNA functionality of the parent and subfragment sequences involves an alteration in pre-mRNA processing or in the stabilization, translational efficiency, localization, sequestration, editing, or splicing functions of the UTR-associated mRNA, more preferably, the alteration in pre-mRNA processing or in the stabilization, translational efficiency, localization, sequestration, editing, or splicing functions of the UTR-associated mRNA comprises a change of at least 20% above or below the corresponding value for a control mRNA that lacks the sequence.

The fourth aspect features a method of identifying a candidate compound having an effect on an RNA/RBP binding pair interaction or mRNA functionality, wherein the method involves contacting an RNA molecule comprising at least one of the nucleic acid sequences of SEQ ID NOs 1-20, or at least one optimized subfragment sequence of SEQ ID NOs 1-20, with at least one RBP, and at least one test compound, and measuring the RNA/RBP binding pair interaction and/or mRNA functionality, wherein a candidate compound is identified as a test compound that affects the interaction and/or functionality. Preferably, the mRNA functionality of the parent or subfragment sequence involves an alteration in pre-mRNA processing or in the stabilization, translational efficiency, localization, sequestration, editing, or splicing functions of the mRNA, more preferably, the alteration in pre-mRNA processing or in the stabilization, translational efficiency, localization, sequestration, editing, or splicing functions of the UTR-associated mRNA comprises a change of at least 20% above or below the corresponding value for a control mRNA that lacks the sequence.

The fifth aspect of the invention provides a method for identifying an RBP that interacts with an RNA molecule comprising the nucleic acid sequence, or an optimized subfragment sequence, of any one of SEQ ID NOs 1-20, wherein the method involves contacting the RNA molecule with at least one RBP, and measuring RNA/RBP binding pair interactions. The detection of an RNA/RBP binding pair interaction identifies the RPB that interacts with the RNA molecule.

By “RNA/RBP binding pair interaction” is meant a physical association between an RNA molecule and an RBP, or an RBP complex made up of more than one protein, that is based on the specific characteristics of the interacting molecules, and is not inhibited by non-specific competitor molecules present at a concentration equivalent to the interacting molecules. The RNA and RBP molecules that form the RNA/RBP binding pair interaction can be separated from their counterpart, non-associated molecules by filter binding assay, electrophoretic mobility assay, homopolymer beads, or fluorescent anisotropy assay.

By “RBP binding activity” is meant the specific binding affinity of an RNA molecule comprising a certain nucleic acid sequence for the RBP with which it forms an RNA/RBP interacting pair. The RBP binding activity is specific for a particular RNA molecule if the RNA binds an RBP or RBP complex with at least 10-fold higher affinity than does a non-relevant RNA molecule with the same G-C content. The affinity measures can be determined by filter binding assay, electrophoretic mobility assay, homopolymer beads, or fluorescent anisotrophy assay.

By “mRNA functionality” is meant the ability of an mRNA molecule comprising a certain nucleic acid UTR sequence in association with a protein's coding region to alter the protein expression from the linked coding region by altering the pre-mRNA processing, or the stabilization, translational efficiency, localization, sequestration, or editing and splicing functions of the mRNA. The mRNA functionality can be assessed, for example, by measuring the half-life of the transcript (Saulnier-Blache et al., Mol. Pharmacol. 50: 1432-1442, 1996; Yang et al., J. Biol. Chem. 272: 15466-15473, 1997), polysomal distribution along the transcript (Izquierdo et al., Mol. Cell. Biol. 17: 5255-5268, 1997; Luis et al., J. Biol. Chem. 268: 1868-1875, 1993; Santaren et al., J. Biochem. 113: 129-131, 1993), or the transcript's intracellular distribution (Yang et al., 1997, supra). Any of the above measures of function for a UTR-associated transcript that differs by 20% or more above or below the value for the corresponding UTR-free transcript indicates that the UTR has mRNA functionality. An increase in the half-life (or a decrease in degradation rate) of the full length transcript indicates an increase in mRNA stability; an increase in transcript length indicates an increase in translational efficiency; and an increase in a transcript's relative distribution in the cytosol indicates in increase in transport out of the nucleus.

By “equivalent to said parent sequence” is meant RBP binding activity or mRNA functionality differing by no more than 50%, preferably, by no more than 30%, and, more preferably, by no more than 10% from the corresponding activity or functionality in the parent sequence.

By a “nucleic acid subfragment” is meant a shorter sequence derived from a longer parent sequence. The subfragment may be shortened by cleaving the 5′ or the 3′ end of the parent, or by internal deletion of a parent sequence, or by any combination of the above.

By an “optimized” nucleic acid sequence is meant a nucleic acid subfragment of a longer parent sequence, wherein the subfragment still maintains an RNA/RBP binding activity and/or mRNA functionality that is equivalent to the parent nucleic acid sequence. An optimized sequence can result from cleavage of the parent sequence at the 5′ and/or 3′ ends, from an internal deletion, or from a combination of the above. Alternatively, such a nucleic acid sequence may be to synthesized.

A nucleic acid molecule or nucleic acid segment referred to as having a specific nucleic acid sequence is intended to mean a nucleic acid molecule in any of its corresponding forms, for example, DNA, cDNA, RNA, or mRNA.

Other features and advantages of the invention will be apparent from the following detailed description, and from the claims.

### DETAILED DESCRIPTION

RNA/RBP binding pair interactions are known to regulate protein expression through the modulation of RNA stabilization, translational efficiency, RNA localization, RNA transcription, and/or RNA editing and splicing processes. Given the importance of RNA/RBP binding pair interactions in the regulation of protein expression, they are attractive targets for the drug screening assays of the present invention. A compound identified in these screens as having an effect on a particular RNA/RBP binding pair interaction, or on the mRNA functionality, can modulate the expression of a gene product that is endogenously associated with the RNA sequence screened. Therefore, such compounds can be administered as a therapeutic for diseases associated with this protein.

We have discovered certain nucleic acid sequences, derived from the mRNA untranslated regions (UTRs) of a variety of therapeutically-relevant genes, that specifically bind RNA binding proteins (RBPs) (SEQ ID NOs 1-20). We have also discovered methods of subdividing such RBP-binding nucleic acid sequences (parent sequences) to obtain shorter nucleic acid sequences that maintain an RNA/RBP binding pair interaction or mRNA functionality that is equivalent to the parent sequences.

The mRNA functionality of the parent or optimized sequences involves to the ability of an mRNA molecule, comprising a certain nucleic acid UTR sequence in association with a protein's coding region, to alter the protein expression from the linked coding region by altering the pre-mRNA processing, or the stabilization, translational efficiency, localization, sequestration, or editing and splicing functions of the mRNA.

The smaller, or optimized sequences, can be used as substitutes for the parent sequences for the purposes further discussed below. If further confirmation of whether the optimized sequence is an adequate substitute for the parent sequence is desired, either the RBP binding activity or the mRNA functionality of the optimized sequence, or both properties, can be assessed to more predictably determine whether the optimized sequence will function in a manner that is relevant to the parent sequence. These optimized sequences have the advantage of having reduced nonspecific protein binding and of isolating one or more of the regulatory activities of the RNA molecules so that therapeutics with specificity for a given activity can be readily identified.

The parent sequences of the present invention, or their optimized subfragments, can be translated into mRNA transcripts for the following: 1) to screen for compounds that affect the RBP binding activity of a particular RNA/RBP binding pair interaction, and/or the mRNA functionality; 2) to identify novel RNA/RBP binding pair interactions; and 3) to modify the protein expression of a heterologous gene. These methods are further discussed below.

### The Parent Sequences

We have identified twenty nucleic acid sequences that possess specific RBP binding ability. The twenty sequences are found in the mRNA UTR regions from eighteen different genes that were initially chosen for study because of their to biological importance. The 5′ and 3′ UTRs were identified from database sequences having the following Accession #'s and encoding the following gene products: the human interleukin-6 receptor, Accession #'s X12830, M20556 (SEQ ID NO 1); human cyclooxygenase-2, Accession #M90100 (SEQ ID NO 0.2 and SEQ ID NO 11); human ubiquitin, Accession #U49869, X04803 (SEQ ID NO 3); human uracil DNA glycosylase-1 (Ung-1), Accession #X89398 (SEQ ID NO 4); human excision repair controlling gene, Accession #U16815 (SEQ ID NO 5); human cytochrome C oxidase 6b, Accession #D28426 (SEQ ID NO 6); human cytochrome C oxidase 5b, Accession #U41284 (SEQ ID NO 7); human Beta-2 adrenergic receptor, Accession #M15169, J02728, M16106 (SEQ ID NO 8); human breast cancer susceptibility-2 (BRCA2), Accession #U43746 (SEQ ID NO 9); human interleukin-4, Accession #M23442 (SEQ ID NO 10); human vascular cell adhesion molecule-1, Accession #M73255 (SEQ ID NO 12 and SEQ ID NO 13); human interleukin-11, Accession #81890 (SEQ ID NO 14); human macrophage CSF receptor (c-fins proto-oncogene), Accession #X03663 (SEQ ID NO 15); human interleukin-9, Accession #M86593, M55519 (SEQ ID NO 16); human leptin (murine obesity homologue), Accession #NM_{—}000230 (SEQ ID NO 17); rat neuropeptide Y5 receptor, Accession #U66274 (SEQ ID NO 18); rat orexin receptor-1, Accession #AF0411244 (SEQ ID NO 19); and mouse mahogany protein, Accession #AF116897 (SEQ ID NO 20). The sequences of the present invention were amplified by PCR from cellular DNA, genomic DNA, or cDNA made from a relevant cell line by isolation of total RNA and reverse transcription.

The RBP binding activity of the UTR sequences was demonstrated as follows. The UTR sequence was placed under the control of a promoter for in vitro transcription, such as an SP6 or T7 promoter, and RNA was produced and to radioactively labeled. The RNA was then mixed with a protein extract from an appropriate cell line that endogenously expresses the UTR-associated gene, for example, U937 and K562 cell lines were used for inflammatory genes, the 3T3-L1 cell line was used for leptin and mahogany genes, and rat brain extract was used for rat neuropeptide Y5 receptor and orexin receptor-1 genes. The protein extract was prepared by lysing cells with digitonin in buffer containing 25 mM Tris, pH 7.9, 0.5 mM EDTA, 0.1 mM PMSF, 2 mM NaF, 2 mM NaPPi, and 1-10 μg/ml of the protease inhibitors aprotinin, leupeptin, and pepstatin. Extracts were centrifuged twice at 16,000×g for 15 minutes at 4 C to remove nuclei, aliquoted, snap frozen on dry ice, and stored at −80 C until use.

The binding pair interactions between the UTR sequences of the invention and RBP's in the cellular extracts were facilitated by a binding solution containing 7.5 mM Bis-Tris Propane, pH 8.5, 50 mM K⁺, 1 mM Mg⁺⁺ 0.2 mM DTT, and 10% (v/v) glycerol. Poly r(G), tRNA, heparin molecules, and unrelated RNA molecules of similar length were used as non-specific competitors of the RNA/RBP binding pair interactions. The interactions were detected by filter binding assay or electrophoretic gel mobility shift assay.

Techniques for in vitro transcription of RNA molecules and methods for cloning genes encoding known RNA molecules are described in the literature (Sambrook et al.) and commercial kits are available (mCap RNA capping kit, Stratagene). Other methods of preparing cell extracts and detecting RNA/RBP binding pair interactions are described in the literature (see, e.g., WO 98/04923) and are further discussed below with regard to the optimization of fragments and screening assays.

### Optimization of Subfragments

Creation of Subfragments

To optimize the parent sequences of SEQ ID NOs 1-20, or other parent UTR-derived nucleic acid sequences, standard restriction mapping techniques are used to create smaller sequences that maintain an RBP binding activity and/or mRNA functionality equivalent to the parent sequences. Alternatively, PCR amplification using appropriate probes or organic synthesis may be used.

In the restriction enzyme approach, the parent nucleic acid sequence is linearized, if necessary, and divided to create subfragments. Different combinations of restriction enzymes are used to digest the parent sequences so that a variety of overlapping sequences are created for each parent sequence. In vitro transcription is carried out to produce mRNA transcripts encoding the subfragments as well as the parent sequences. These transcripts are used for testing RBP binding activity and/or mRNA functionality. Subfragments, or combinations of subfragments, which are identified as having RBP binding activity and/or RNA functionality equivalent to the parent sequences, are optimized, and can be further digested such that smaller and smaller optimized subfragments are identified.

RNA/RBP Binding Pair Interactions

The RBP binding activity of the nucleic acid subfragments can be assessed and compared to their respective parent sequences by measuring the RNA/RBP binding pair interaction for each of the nucleic acid sequences. Subfragments with RBP binding activity equivalent to the parent sequence are considered to be optimized by this process. The methods of detecting such RNA/RBP binding pair interactions are well known in the art, and include, for example, filter binding assays (Wu and Uhlenbeck, Biochemistry 26: 8221-8227, 1987; Carey and Uhlenbeck, Biochemistry 22: 2610-2615, 1983), electrophoretic gel mobility shift assays (Izquierdo and Cuezva, Mol. Cell. Biol. 17:5255-5268, 1997; Malter, Science 246: 664-666, 1989; Zaidi and Malter, J. Biol. Chem. 269: 24007-24013, 1994; Claffey et al., Mol. Biol. Cell 9: 469-481, 1998; Brewer, Mol. Cell. Biol. 11: 2460-2466, 1991); homopolymer beads (Siomi et al., Cell 77: 33-39, 1994), or fluorescence anisotrophy (Tetin et al., Biochemistry 32: 9011-9017, 1993; Goss et al., Nucleic Acids Research 11: 5589-5602, 1983; and Liang et al., WO 98/39484).

It is preferred that conditions allowing detection of interactions between nearly every type of RNA and RBP pair be employed. Exemplary protocols, binding conditions, and RNA binding proteins that may be used are disclosed in detail in PCT application WO 98/04923 and are summarized below.

The preferred conditions allow detection of a majority of RNA/RBP interactions. The interactions are facilitated in a binding solution that includes a buffer, a monovalent cation, a divalent cation, a reducing agent, and a density agent. The basic method includes forming a binding solution containing the RNA molecules and binding buffer, heating the solution to denature the RNA, cooling the solution to the reaction temperature to fold the RNA in proper formation, adding RBPs, and detecting the interactions using any suitable procedure. The specificity of binding pair interactions is assessed by comparing the binding in the presence of specific and nonspecific competing RNA. If desired, a competitor of nonspecific RNA/RBP interactions, such as poly r(G), tRNA, heparin, or unrelated RNA molecules of similar length can be added to the binding solution to reduce the background of nonspecific binding. It is preferred that detection involve the separation of interacting RNA molecules and RBPs, such as on the basis of size or physical properties. Two preferred methods are filter binding and gel mobility shift.

Detection of interactions between RNA binding proteins and RNA molecules can be facilitated by attaching a detectable label to the RNA molecule. Generally, labels known to be useful for nucleic acids can be used to label RNA molecules, including, for example, isotopes such as ³³P, and ³⁵S, fluorescent labels such as fluorescein (FITC), 5,6-carboxymethyl fluorescein, Texas red, nitrobenz-2-oxa-1,3-diazol-4-yl(NBD), coumarin, dansyl chloride, rhodamine, 4′-6-diaminidino-2-phenylinodole (DAPI), the cyanine dyes (Cy3, Cy3.5, Cy5, Cy5.5, and Cy7), and biotin.

Labeled nucleotides are the preferred form of label since they can be directly incorporated into the RNA molecules during synthesis. Examples of labeled nucleotides include BrdUrd (Hoy and Schimke, Mutation Research 290: 217-230 (1993)), BuUTP (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res. 22:3226-3232 (1994)). A preferred nucleotide analog label for RNA molecules is Biotin-14-cytidine-5′-triphosphate. Fluorescein, CY3, and Cy5 can be linked to dUTP for direct labeling. Cy3.5 and Cy7 are available as avidin or anti-digoxygenin conjugates for secondary detection of biotin- or digoxygenin-labeled probes.

The RBPs used for optimization can be part of a crude or purified cellular or nuclear extract, and can be used either in isolation or in combination. These RBPs can be prepared using known methods of protein extraction and purification (Ashley et al., Science 262: 563-566, 1993; Rouault et al., Proc. Nat. Acad. Sci. USA 86: 5768-5772, 1989; Neupert et al., Nucleic Acids Research 18: 51-55, 1990; Zhang et al., Mol. Cell. Biol. 13: 7652-7665, 1993; and Burd and Dreyfuss, Science 265: 615-21, 1994). Alternatively, known RBPs can be produced recombinantly using standard techniques. DNA encoding RNA binding proteins can be obtained from available clones, by synthesizing a DNA molecule encoding an RNA binding protein with a known amino acid sequence, or by cloning the gene encoding the RNA binding protein. Techniques for recombinant expression and methods for cloning genes encoding known proteins are well known (Sambrook et al., Molecular Cloning, Cold Spring Harbor Laboratory, 1989).

Detection of interactions between RNA binding proteins and RNA molecules can also be facilitated by attaching a detectable label to the RBP. Preferred labels include ¹²⁵I, ³H, ³⁵S, and, in the case of recombinant proteins, they can be incorporated through the use of labeled amino acids. Techniques for labeling and detecting proteins are known in the art (Sambrook et al. and Ausubel et al., Current Protocols in Molecular Biology, John Wiley & Sons, Inc., 1996). Detection of an RBP can also be achieved by the use of an RBP specific antibody (Johnstone and Thorpe, Immunochemistry in Practice, Blackwell Scientific Publications, 1997).

Functionality

The mRNA functionality of the subfragment sequences, as compared to their corresponding parent sequences, can be used as an independent tool for identifying optimized subfragments with function equivalent to their parent sequences. Alternatively, the mRNA functionality studies can be used in combination with RNA/RBP binding pair interaction studies to further verify that the optimized sequences are adequate substitutes for the parent sequences. Studies of mRNA functionality involve, for example, assessments of the sequences' effects on translational efficiency, mRNA stability, and transcript intracellular transport, as well as the editing and splicing of RNA.

To determine whether a parent sequence regulates mRNA functionality, or whether a subfragment sequence regulates functionality in a fashion equivalent to the parent, constructs are generated in which the parent or subfragment UTR sequence to be tested is adjoined to the coding segment of a gene at a position upstream or downstream of the coding region, depending on whether the UTR is derived from a 5′ or 3′ UTR, respectively. The coding region may encode a reporter gene, or the gene that is endogenously associated with the UTR sequence with an inserted tag, such as an epitope tag. Intracellular expression of the construct can be achieved by transfecting cells with expression vectors containing the construct. If the parent or subfragment sequence is adjoined to a reporter gene, then changes in the level of reporter gene product can be used as an indication that the parent or subfragment sequence regulates at least one aspect of mRNA functionality.

When assessing the functionality of a parent sequence, transcripts from constructs which contain the coding region, but lack the UTR sequence, are used as controls. To determine whether a subfragment maintains a function equivalent to the parent sequence, transcripts from a construct containing the full-length parent sequence are used as controls.

mRNA Stability

The stability of mRNA can be determined using either in vivo or in vitro transcribed mRNA. In cells, stability is determined by first blocking transcription in transfected cells with a compound such as actinomycin D (5 μg/ml), and then measuring the degradation rate of the transcripts by quantitating their level in cells harvested at different times. To quantify the level of transcript, total cell RNA purified from harvested cells is subjected to electrophoresis followed by transfer to a filter by pressure blotting. Following incubation, the filter is subject to hybridization by a radiolabeled probe designed to detect the transcript sequence. Additionally, real time PCR with total cell RNA can be used for quantitating mRNA degradation rates. Such degradation rates are calculated, for example, by densitometric scanning of the autoradiographs (Saulnier-Blache et al., Mol. Pharmacol. 50: 1432-42, 1996; Yang et al., J. Biol. Chem. 272: 15466-15473, 1997). A decrease in the rate of degradation indicates an increase in mRNA stability.

Translational Efficiency

A reticulocyte system can be used to assess the effect of the UTR RNA sequences on translational efficiency. In vitro synthesized mRNAs, derived from corresponding plasmids (up to 40 μg/ml) are utilized as templates for protein synthesis in a nuclease-treated rabbit reticulocyte lysate (Amersham). The reactions are performed in the presence of 40 μCi of L-[³⁵S]methionine (greater than 1 Ci/mM) and 40 U of RNasin (Izquierdo et al., Mol. Cell. Biol. 17: 5255-5268, 1997; Luis et al., J. Biol. Chem. 268: 1868-1875, 1993; Santaren et al., J. Biochem. 113: 129-131, 1993). At various times for up to 1 hour, the products are separated by SDS-PAGE. Ribosomal distribution is indicated by the length (migration) of the transcripts; an increase in length indicates an increase in translational efficiency. Efficiency is also assessed by monitoring the amount of protein produced; an increase in amount indicates an increase in translational efficiency. In addition, translational efficiency can be monitored by analyzing the distribution of the transcripts on polysomes. Transcripts associated with high molecular weight polysomes represent actively transcribed messages, those on low molecular weight polysomes are associated with poor translation, and those found in the nonpolysome fraction of a gradient are not being translated. By analyzing the percentages associated with each fraction, an estimate of translational efficiency can be determined.

Transcript Distribution

The poor processing of transcripts may reflect failure of the transcript to move from the nucleus to associate with translationally active ribosomes in the cytoplasm. To assess the effect of the parent UTR sequences and the subfragments on this function, the cytosolic versus total cellular transcript concentration is compared in cells transfected. Harvested cells are lysed and subjected to sucrose gradient fractionation. RNA is precipitated from cell fractions, denatured, and blotted onto a nylon membrane in a slot-blot apparatus. Following hybridization to a labeled probe, the transcript RNA levels are quantitated for the various fractions. Alternatively, constructs with or without the UTR sequence can be in vitro labeled with a fluorescent tag and transfected into the cell. Cellular distribution of the transcript is then analyzed using a fluorescent microscope. If the relative quantity of transcript in the cytoplasm compared to total cell transcript RNA is modified when a UTR sequence is present, then this UTR sequence affects cytoplasmic transport of its associated transcript.

### Screening Assays

RNA molecules comprised of the disclosed parent nucleic acid sequences, or their optimized subfragments, can be used in screening procedures to identify compounds that affect the RNA/RBP binding pair interactions associated with these RNA molecules. In a similar way, novel RNA/RBP binding pair interactions associated with these RNA molecules can be identified. As an alternative to, or in conjunction with screening for RNA/RBP binding pair interactions, screening procedures can be tailored to identify compounds that alter the mRNA functionality of the RNA molecules. The screening protocols can be designed to allow simultaneous assessment of the effect of numerous test compounds in a high throughput screening assay, as described in further detail in PCT application WO 98/04923.

Use of the longer parent sequences provides the benefit of more physiologically relevant target sequences. On the other hand, use of the optimized sequences provides the advantage of a smaller alternative molecules that can be used when they are better suited to the assay methodology. For example, when a large RNA molecule is used in an RBP binding assay, actual RBP binding may be difficult to detect because the RNA/RBP binding pair is only slightly larger than the RNA molecule alone. In addition, nonspecific interactions with other parts of the parent sequence transcript can interfere with detection of specific interactions. This problem can be alleviated by using a smaller, optimized subfragment, which has reduced nonspecific binding and an equivalent RBP binding activity that is easier to detect because the interacting RNA/RBP complex is significantly larger than the RNA alone. Furthermore, the optimized sequences provide more specific information regarding the actual nucleic acid site of RNA/RBP binding pair interaction, which facilitates the rational drug design of compounds that affect this interaction.

The screening protocols for identifying compounds that affect RNA/RBP binding pair interactions include, for example, filter binding assays (Wu and Uhlenbeck, Biochemistry 26: 8221-8227, 1987; Carey and Uhlenbeck, Biochemistry 22: 2610-2615, 1983), electrophoretic gel mobility shift assays (Izquierdo and Cuezva, Mol. Cell. Biol. 17:5255-5268, 1997; Malter, Science 246: to 664-666, 1989; Zaidi and Malter, J. Biol. Chem. 269: 24007-24013, 1994; Claffey et al., Mol. Biol. Cell 9: 469-481, 1998; Brewer, Mol. Cell. Biol. 11: 2460-2466, 1991); homopolymer beads (Siomi et al., Cell 77: 33-39, 1994), or fluorescence anisotrophy (Tetin et al., Biochemistry 32: 9011-9017, 1993; Goss et al., Nucleic Acids Research 11: 5589-5602, 1983; and Liang et al., WO 98/39484) (see generally, WO 98/04923).

With regard to mRNA functionality, compounds are screened for the ability to alter the RNA molecule's pre-mRNA processing, or stabilization, translational efficiency, localization, sequestration, or editing and splicing functions of the mRNA. These functions can be assessed, for example, by measuring the expression of the UTR-associated coding region, the half-life of the transcript (Saulnier-Blache et al., Mol. Pharmacol. 50: 1432-1442, 1996; Yang et al., J. Biol. Chem. 272: 15466-15473, 1997), polysomal distribution along the transcript (Izquierdo et al., Mol. Cell. Biol. 17: 5255-5268, 1997; Luis et al., J. Biol. Chem. 268: 1868-1875, 1993; Santaren et al., J. Biochem. 113: 129-131, 1993), the type of polysome associated with the transcript, or the transcript's intracellular distribution (Yang et al., 1997, supra).

In general, extracts, compounds, or chemical libraries that can be used in screening assays are known in the art. Examples of such extracts or compounds include, but are not limited to, extracts based on plant, fungal, prokaryotic, or animal sources, fermentation broths, and synthetic compounds, as well as modification of existing compounds. Numerous methods are also available for generating random or directed synthesis (e.g. semi-synthesis or total synthesis) of any number of chemical compounds, including, but not limited to, saccharide-, lipid-, peptide-, and nucleic acid-based compounds. Libraries of genomic DNA or cDNA may be generated by standard techniques (see, e.g., Ausubel et al., supra) and are also commercially available (Clontech Laboratories Inc., Palo Alto. CA). Nucleic acid libraries used to screen for compounds that alter RNA/RBP binding pair interactions or mRNA functionality are not limited to the species from which the RNA or RBP is derived. For example, a Xenopus cDNA may be found to encode a protein that alters a human RNA/RBP interaction.

Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceanographics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods.

When a crude extract is found to modulate an RNA/RBP binding pair interaction or mRNA functionality, further fractionation of the positive lead extract is necessary to isolate chemical constituents responsible. Thus, the goal of the extraction, fractionation, and purification process is the characterization and identification of a chemical entity within the crude extract having the interaction- or function-modulating activities. The same assays described herein for the detection of interactions in mixtures of compounds can be used to purify the active component and to test derivatives thereof. Methods of fractionation and purification of such heterogenous extracts are known in the art. If desired, compounds shown to be useful agents for treatment are chemically modified according to methods known in the art.

Compounds which modulate an RNA molecule's RNA/RBP binding pair interaction or mRNA functionality may be administered by any appropriate route for treatment or prevention of a disease or condition associated with the expression of the protein endogenously associated with the gene from which SEQ ID NOs 1-20 are derived. Examples of such diseases and conditions include neurodegenerative disease, stroke, cardiovascular disease, peripheral vascular disease, high blood pressure, cancer, including breast cancer, inflammatory diseases, such as rheumatoid arthritis, Crohn's disease, diseases associated with cellular proliferation, metabolic disorders, such as obesity and diabetes, and infectious diseases, such as bacterial or viral infections. Administration may be parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington's Pharmaceutical Sciences.” Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

The formulations can be administered to human patients in therapeutically effective amounts (e.g., amounts which prevent, eliminate, or reduce a pathological condition) to provide therapy for a disease or condition. Typical dose ranges are from about 0.1 μg/kg to about 1 g/kg of body weight per day. The preferred dosage of drug to be administered is likely to depend on such variables as the type and extent of the disorder, the overall health status of the particular patient, the formulation of the compound excipients, and its route of administration.

An additional use for the parent UTR sequences or their optimized subfragments is their incorporation into a recombinant construct such that expression of the construct is controlled by the UTR sequence. For example, a nucleic acid sequence of the invention can be inserted into a heterologous gene to form all or a part of the untranslated region of the gene's mRNA transcript. It is expected that the UTR sequence will interact with an RBP in these recombinant RNA molecules and that protein expression of the heterologous gene will be affected. This is analogous to recombining promoters with heterologous coding regions to alter or control the expression of the coding region.

### Other Embodiments

All publications and patent applications mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art. Other embodiments are within the claims.

What is claimed is:

1. A nucleic acid sequence comprising any one of the nucleic acid sequences of SEQ ID NOs 1-20, or a subfragment nucleic acid sequence derived from any one of the sequences of SEQ ID NOs 1-20, wherein an mRNA molecule comprising said sequence has RNA binding protein (RBP) binding activity or regulates the functionality of said mRNA.

2. The nucleic acid sequence of claim 1, wherein said subfragment nucleic acid sequence is optimized.

3. The nucleic acid sequence of claim 1, wherein the regulation of mRNA functionality comprises an alteration in pre-mRNA processing or in the stabilization, translational efficiency, localization, sequestration, editing, or splicing functions of said mRNA.

4. A method of identifying an optimized subfragment of any one of the parent nucleic acid sequences of SEQ ID NOs 1-20, said method comprising isolating a subfragment nucleic acid sequence from said parent nucleic acid sequence, assaying RNA molecules comprising said subfragment for RBP binding activity or mRNA functionality, and identifying a subfragment nucleic sequence that maintains an RBP binding activity and/or mRNA functionality that is equivalent to said parent sequence.

5. The method of claim 4, wherein said subfragment nucleic acid sequence is isolated by restriction enzyme digestion.

6. The method of claim 4, wherein said subfragment is identified by deletion mapping.

7. The method of claim 4, wherein said mRNA functionality comprises an alteration in pre-mRNA processing or in the stabilization, translational efficiency, localization, sequestration, editing, or splicing functions of said mRNA.

8. A nucleic acid sequence identified as an optimized subfragment of any one of SEQ ID NOs 1-20 by the method of claim 4.

9. A method of identifying a candidate compound having an effect on an RNA/RBP binding pair interaction or mRNA functionality, said method comprising contacting an RNA molecule comprising at least one nucleic acid sequence of any one of SEQ ID NOs 1-20, or at least one optimized subfragment sequence derived from any one of SEQ ID NOs 1-20, with at least one RBP, and at least one test compound, and measuring said RNA/RBP binding pair interaction and/or mRNA functionality, wherein a candidate compound is identified as a test compound that affects said interaction and/or functionality.

10. The method of claim 9, wherein said mRNA functionality comprises an alteration in pre-mRNA processing or in the stabilization, translational efficiency, localization, sequestration, editing, or splicing functions of said mRNA.

11. A method for identifying an RBP that interacts with an RNA molecule comprising the nucleic acid sequence of any one of SEQ ID NOs 1-20, or an optimized subfragment sequence of any one of SEQ ID NOs 1-20, said method comprising contacting said RNA molecule with at least one RBP, and measuring RNA/RBP binding pair interactions, wherein detection of said interactions identifies said RBP that interacts with said RNA molecule.


SEQUENCE LISTING (20 sequences)

SEQ ID NO: 1 - DNA, 278 bp, Homo sapiens
ctgggtaactagggaagataatattaaggaagacaatgtgaaaagaaaaatgagcctggcaagaatgcgtttaaacttggtttttaaaaaactgctgactgttttctcttgagagggtggaatatccaatattcgctgtgtcagcatagaagtaacttacttaggtgtgggggaagcaccataactttgtttagcccaaaaccaagtcaagtgaaaaaggaggaagagaaaaaatattttcctgccaggcatggaggcccacgcacttcgggaggtcg

SEQ ID NO: 2 - DNA, 243 bp, Homo sapiens
gaagtaactaatgtttgaaattttaaagtacttttgggtatttttctgtcatcaaacaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaag

SEQ ID NO: 3 - DNA, 122 bp, Homo sapiens
gggcggttggctttgttgggtgagcttgtttgtgtccctgtgggtggacgtggttggtgattggcaggatcctggtatccgctaacagaactaggtcaaaatgcagatcttcgtgaaaaccc

SEQ ID NO: 4 - DNA, 127 bp, Homo sapiens
ctcccagcccgtctccccgctccagtttagaacctaattcccaattcccggaccgggcccagccctgggctcttactgtccgcttttgctgggacctgttccacaaatgggcgtcttctgccttggg

SEQ ID NO: 5 - DNA, 190 bp, Homo sapiens
agcgcccgggcaggccaccccgagccccttaactgcgcaggcgctctcactcagaaaggccgctgggtgcgggagcgcagaggcggtgcagggcggctggctcgcctcggcgtgcagtgcgcgtgcgtggagctgggagctaggtcctcggagtgggccagagatggcggcggccgacggggctttgccg

SEQ ID NO: 6 - DNA, 69 bp, Homo sapiens
ttctttgctgagggtcacattgagctgcaggttgaatccggggtgcctttaggattcagcaccatggcg

SEQ ID NO: 7 - DNA, 84 bp, Homo sapiens
aagtccctcctgtctctgcagcttgttcccggaagttttgctgctagtcgcggacgcaatggcttcaaggttacttcgcggagc

SEQ ID NO: 8 - DNA, 230 bp, Homo sapiens
actgcgaagcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgcttacctgccagactgcgcgccatggggcaacc

SEQ ID NO: 9 - DNA, 234 bp, Homo sapiens
gtggcgcgagcttctgaaactaggcggcagaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgccta

SEQ ID NO: 10 - DNA, 260 bp, Homo sapiens
ggaaaggctaaagacgatcatgagagagaaatattcaaagtgttcgagctgaatattttaatttatgagtttttgatagctttattttttaagtatttatatatttataactcatcataaaataaagtatatatagaatctaacagcaatggcatttaatgtattggctatgtttacttgacaaatgaaattatggtttgcaacttttagggaaatcaatttagtttaccaagagactataaatgctatggagccaaaac

SEQ ID NO: 11 - DNA, 159 bp, Homo sapiens
gtccaggaactcctcagcagcgcctccttcagctccacagccagacgccctcagacagcaaagcctacccccgcgccgcgccctgcccgccgctgcgatgctcgcccgcgccctgctgctgtgcgcggtcctggcgctcagccatacagcaaatccttg

SEQ ID NO: 12 - DNA, 124 bp, Homo sapiens
actccgcggtatctgcatcgggcctcactggcttcaggagctgaataccctcccaggcacacacaggtgggacacaaataagggttttggaaccactattttctcatcacgacagcaacttaaa

SEQ ID NO: 13 - DNA, 262 bp, Homo sapiens
taaaatagcaacactctatatttagattgttaaaataactagtgttgcttggactattataatttaatgcatgttaggaaaatttcacattaatatttgctgacagctgacctttgtcatctttcttctattttattccctttcacaaaattttattcctatatagtttattgacaataatttcaggttttgtaaagatgccgggttttatatttttatagacaaataataagcaaagggagcactgggttgactttcaggt

SEQ ID NO: 14 - DNA, 253 bp, Homo sapiens
cgaggaggaggggactggggtcccggattcttgggtctccaagaagtctgtccacagacttctgccctggctcttccccatctaggcctgggcaggaacatatattatttatttaagcaattacttttcatgttggggtggggacggaggggaaagggaagcctgggtttttgtacaaaaatgtgagaaacctttgtgagacagagaacagggaattaaatgtgtcatacatatccacttgagggcgatttgt

SEQ ID NO: 15 - DNA, 285 bp, Homo sapiens
gagccctcaccccccgcctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctc

SEQ ID NO: 16 - DNA, 210 bp, Homo sapiens
gatgagaggcaagatatgaagatgaaatattatttatcctatttattaaatttaaaaagctttctctttaagttgctacaatttaaaaatcaagtaagctactctaaatcagtatcagttgtgattatttgtttaacattgtatgtctttattttgaaataaatacatatgtggaaaaaacaacatgagctggtctcttggcaattattc

SEQ ID NO: 17 - DNA, 239 bp, Homo sapiens
ctggtttcatttctactgtgactgatgttacatcacagtgtttgcaatggtgttgccctgagtggatctccaaggaccaggttattttaaaaagatttgttttgtcaagtgtcatatgtaggtgtctgcacccaggggtggggaatgtttgggcagaagggagaaggatctagaatgtgttttctgaataacatttgtgtggtgggttctttggaaggagtgagatcattttcttatct

SEQ ID NO: 18 - DNA, 340 bp, Rattus norvegicus
cctattagtgaagattggtaaaattgtcagtttaacccggctgtcctactactaatatttaatttttcaaatatgaaaaggtttcagattttgtttagatttatatcacattaaacactgtcaaataaaggctgtttttatatgcatcgttgatgttccaaaatgtgaagtctaaatggtgtctgtatttccaattattaaataacttctaagatcatttttaaaagtctgtagatggtatggatagctagttgtttgttaatataaagtaaaagtagatagctgatttatgttgtacctatgtcgtatgtatattaggtatcgtgttgtctcactaaag

SEQ ID NO: 19 - DNA, 240 bp, Rattus norvegicus
gtcgacgtgtcgaaaggatgctcaggatatgaatgagccagcggacgcgtgaatggaggtggatggaagcgtgaagtacaaggcggggctccagctgggtgacctcgaggcagacaggccggggctccctcccctctcctcccagcctcccactccgctcccctccccgcggcgcgcagaccttccgagccctcccgagctaccgcgggcaacagagacgggaccacgcagccaccgagc

SEQ ID NO: 20 - DNA, 259 bp, Mus musculus
gccctctgtcagcagctctccagcatggtcctctgacactcctcagatgaactgttctcatcggaagcttgctgtctttttacaagatgagcttttactctcttccaggaagtagctttttttctagctgagaattaataatggtctttctctttggaagtcatatcaaagtataattgatgggggccttgttttgttttggtttttggagacagggtctcactgtgtagtcctagctggcctggaactcactatgtag